(19) United States
(12) Patent Application Publication
(10) Pub. No.: US 2023/0293563 A1
(43) Pub. Date: Sep. 21, 2023

(54) TREATMENT FOR ADVANCED COLORECTAL CARCINOMA

(71) Applicant: NEOCAGE CL, Canillo (AD)

(72) Inventor: Manuel Gidekel, Canillo (AD)

(21) Appl. No.: 18/052,091

(22) Filed: Nov. 2, 2022

Related U.S. Application Data
(63) Continuation-in-part of application No. 16/275,531, filed on Feb. 14, 2019, now Pat. No. 11,534,449.

(51) Int. Cl. A61K 31/7048; A61K 38/20; A61P 35/00; C12N 15/86

(52) U.S. Cl. CPC A61K 31/7048; A61K 38/208; A61P 35/00; C12N 15/86; C12N 2710/10043

(57) ABSTRACT

Methods and compositions are provided to treat colorectal tumors. Tricin 7-O-β-D-glucopyranoside (Antartina) in combination with AdIL-12 is shown to be effective in preventing or inhibiting growth of colorectal tumors as well as in eradication of the tumors. Method, and treatment for treating colorectal tumors are suggested.

### CROSS-REFERENCE TO RELATED APPLICATIONS

This application is a continuation-in-part application of U.S. patent application Ser. No. 16/275,531 filed on Feb. 14, 2019, the contents of which is incorporated herein by reference.

### FIELD OF THE INVENTION

The molecules disclosed herein are tricin derivatives useful for treatment of cancers, such as colorectal carcinoma (CRC) and others. The molecules are derivatives of tricin 7-O-β-D-glucopyranoside (also called “Antartina”). The molecules provide an immune response against CRC as shown by CTL assay, analyzing dendritic cell activation and antitumor T cell sub-populations, and tumor re-challenge experiments. Synergistic effects are shown with gene immune therapy. The tricin derivatives are also shown to induce a potent specific cytotoxic T cell response against CRC and long lasting antitumor activity. These tricin derivatives are therefore useful in methods of treating and/or prevent occurrence of CRC and other cancers in mammals.

### BACKGROUND

Colorectal carcinoma (CRC) is a major cause of cancer-related morbidity and mortality worldwide and is responsible for nearly 700,000 deaths each year. (Torre et al., “Global cancer statistics,” CA Cancer J. Clin., 65(2):87-108, 2015). For metastatic disease, surgery is a potential curative option, but less than 20-30% of patients are suitable for resection due to clinical or technical issues, including extra-hepatic disease. (Zampino et al., Crit. Rev. Oneal. Hematol., 2016, 108:154-63). In the last few years, multidisciplinary approaches, such as chemotherapy regimens, radio embolization, and targeted therapies, have been applied to improve patient survival, but as noted above there still remains a significant mortality rate. (Elias et al., Liver Cancer, 2016, 6(1):90-8). Therefore, new therapeutic therapies are needed for patients with a more advanced stage of the disease (Prieto et al., Surg. Clin. North Am., 2004, 84(2):673-96).

With its unique weather and environmental characteristics, Antarctica is home to an extraordinary variety of extremophile organisms including vascular plants such as Deschampsia Antarctica. (Alberti et al., Physiol. Plant., 2002, 115(4):479-86). There are several examples of novel psychrophilic enzymes and new molecules isolated from organisms found in Antarctica with potential uses in medicine. (Cavicchioli et al., Curr. Opin. Biotechnol., 2002, 13(3):253-61). In particular, Deschampsia antarctica is able to tolerate high UV exposure due to the production of secondary metabolites as photoprotector agents, especially flavonoid-like molecules. (Bravo et al., J. Exp. Bot., 2005, 56(414):1189-96, and van de Staaij et al., J. Photochem. Photobiol. B, 2002, 66(1):21-9).

The reported antitumor activities of flavonoids involve the inhibition of proliferation and induction of apoptosis, suppression of protein tyrosine kinase activity, and anti-angiogenic effects. (Middleton et al., Pharmacol. Rev., 2000, 52(4):673-751). However, little is known about the role of tricin derivatives and their bioactivity as anticancer molecules. Tricin 7-O-β-D-glucopyranoside (Antartina) was isolated from the Antarctic plant Deschampsia antarctica and has been previously reported from other plants. (Hidalgo et al., Mol. Cancer Ther., 2011, 10(8):1311-6, and Rubio-Viqueira et al., Clin. Cancer Res., 2006, 12(15):4652-61).
Due to low natural availability, a synthesis was conducted. Various observations have been reported on the antitumor activity of tricin7-O-β-D-glucopyranoside produced synthetically. (See, U.S. Pat. No. 10,058,582, PCT Publication No. WO 2009/064480, and Malvicini et al., Mol. Cancer Therap., 2018, 17(5):966-976).

There is a continuous need for novel and more efficient cancer treatments. Accordingly, this disclosure provides compounds and methods to treat colorectal tumors.

### SUMMARY

Here, it is shown that tricin 7-O-β-D-glucopyranoside (Antartina) and its_derivatives, such as acetylated tricin 7-O-β-D-glucopyranoside, in combination with immune-gene therapy using an adenovirus encoding IL-12, induces a potent antitumoral response against CRC in mice when combined with AdIL-12. In addition to slowing down growth rate of colorectal tumors, combination treatment with 7-O-β-D-glucopyranoside and AdIL-12 (an adenovirus encoding IL-12 genes) is shown to eradicate colorectal tumors in 80% of the cases. It is also disclosed that acetylated tricin 7-O-β-D-glucopyranoside has a superior antitumoral effect in comparison with native tricin 7-O-β-D-glucopyranoside (Antartina) in mice with CRC. Thus, disclosed herein, it is revealed that tricin 7-O-β-D-glucopyranoside (Antartina) in combination with immuno-gene therapy (adenovirus-IL12) shows a synergistic antitumoral effect 80% remission. Additionally, analogues or derivatives of tricin-7-O-β-D-glucopyranoside are shown to have a potent effect on hepatocarcinoma cells.

Disclosed are methods and compositions for treating cancer in a subject in need thereof. The methods described herein comprise administering a pharmaceutically effective composition comprising one or more of tricin 7-O-β-D-glucopyranoside and its derivatives. The one or more derivatives of tricin 7-O-β-D-glucopyranoside are shown in FIG. 1. The derivatives are one or more of: acylated tricin 7-O-β-D-glucopyranoside (6a), [3,4,5-triacetoxy-6-[5-hydroxy-2-(4-hydroxy-3,5-dimethoxy-phenyl)-4-oxo-chromen-7-yl]oxy -tetrahydropyran-2-yl]methyl acetate (5b), 5-hydroxy-2-(4-hydroxy-3,5-dimethoxy-phenyl)-7-[3,4,5-trihydroxy-6 (hydroxymethyl)tetrahydropyran-2-yl]oxy-chromen-4-one (1b), xylose tricin 7-O-β-D-glucopyranoside (1c), 7-[(3R,4R,5S,6R)-3,4-dihydroxy-6-(hydroxymethyl)-5-[(3R,4S,5R,6R)-3,4,5-trihydroxy-6-(hydroxymethyl)tetrahydropyran-2-yl]oxy-tetrahydropyran-2-yl]oxy-5-hydroxy-2-(4-hydroxy-3,5-dimethoxy-phenyl)chromen-4-one (1d), 7-[(3R,4R,5S,6R)-3,4-dihydroxy-6-(hydroxymethyl)-5-[(3R,4S,5S,6R)-3,4,5-trihydroxy-6-(hydroxymethyl)tetrahydropyran-2-yl]oxy-tetrahydropyran-2-yl]oxy-5-hydroxy-2-(4-hydroxy-3,5-dimethoxy-phenyl)chromen-4-one (1e), and 7-[(2S,3R,4R,5S,6R) -3,4-dihydroxy-6-(hydroxymethyl)-5-[(2S,3R,4S,5S,6R)-3,4,5-trihydroxy-6-(hydroxymethyl)tetrahydropyran-2-yl]oxy-tetrahydropyran-2-yl]oxy-5-hydroxy-2-(4-hydroxy-3,5-dimethoxy-phenyl)chromen-4-one. In certain embodiments of the described methods, the cancer is colorectal cancer or hepatocellular carcinoma. In such methods, the subject is any mammal, but is in some embodiments a human.

In certain embodiments of the described methods, the colorectal cancer is an adenocarcinoma, a gastrointestinal carcinoid tumor, a colorectal lymphoma, a gastrointestinal stromal tumor, a leiomyosarcoma, or a melanoma.

In a particular embodiment of the disclosed methods of treating cancer, the one or more derivatives of tricin 7-O-β-D-glucopyranoside is acylated tricin 7-O-β-D-glucopyranoside.

In the presently disclosed methods, the treating of cancer comprises any or all of reducing the proliferation of cancer cells in the subject, reducing the size of one or more tumors in the subject, eradicating the tumor, reducing the risk of mortality of the subject from cancer, or extending the life of the subject.

In some embodiments of the described methods, the method further comprises administering to the subject one or more additional antitumor agents in a combination anticancer therapy. In certain such embodiments, the one or more additional antitumor agents is one or more checkpoint inhibitors. The one or more antitumor agents are, for example, one or more monoclonal antibodies.

Also described herein are several derivatives of tricin 7-O-β-D-glucopyranoside, including acylated tricin 7-O-β-D-glucopyranoside (6a), [3,4,5-triacetoxy-6-[5-hydroxy-2-(4-hydroxy-3,5-dimethoxy-phenyl)-4-oxo-chromen-7-yl]oxy-tetrahydropyran-2-yl]methyl acetate (5b), 5-hydroxy-2-(4-hydroxy-3,5-dimethoxy-phenyl)-7-[3,4,5-trihydroxy-6 hydroxymethyl)tetrahydropyran-2-yl]oxy-chromen-4-one (1b), xylose tricin 7-O-β-D -glucopyranoside (1c), 7-[(3R,4R,5S,6R)-3,4-dihydroxy-6-(hydroxymethyl)-5-[(3R,4S,5R,6R)-3,4,5-trihydroxy-6-(hydroxymethyl)tetrahydropyran-2-yl]oxy-tetrahydropyran-2-yl]oxy-5-hydroxy-2-(4-hydroxy-3,5-dimethoxy-phenyl)chromen-4-one (1d), 7-[(3R,4R,5S,6R)-3,4-dihydroxy-6-(hydroxymethyl)-5-[(3R,4S,5S,6R)-3,4,5-trihydroxy-6-(hydroxymethyl)tetrahydropyran-2-yl]oxy-tetrahydropyran-2-yl]oxy-5-hydroxy-2-(4-hydroxy-3,5-dimethoxy-phenyl)chromen-4-one (1e), and 7-[(2S,3R,4R,5S,6R)-3,4-dihydroxy-6-(hydroxymethyl)-5-[(2S,3R,4S,5S,6R)-3,4,5-trihydroxy-6-(hydroxymethyl)tetrahydropyran-2-yl]oxy-tetrahydropyran-2-yl]oxy-5-hydroxy-2-(4-hydroxy-3,5-dimethoxy-phenyl)chromen-4-one (see FIG. 1).

Thus, disclosed herein are compositions comprising the derivatives of tricin 7-O-β-D-glucopyranoside. Such compositions comprise one or more of these derivative compounds. In certain embodiments, the compositions further comprise one or more pharmaceutically acceptable excipients, as are known in the art to be useful for administering such compositions to a subject in need thereof.

Also disclosed are methods of adoptive T cell therapy (ACT). Such methods comprise various steps including removing dendritic cells from the subject, isolating the dendritic cells, exposing the dendritic cells to the compositions described herein comprising one or more derivatives of tricin 7-O-β-D-glucopyranoside to activate the dendritic cells, and injecting the activated dendritic cells into the subject, thereby generating an immunoprotective response in the subject. In some embodiments the subject from which the dendritic cells were obtained is the same subject into which the activated dendritic cells are injected.

In such ACT methods, the subject in need thereof, in some embodiments, has or has been diagnosed with, or is at risk of cancer. In certain embodiments of such ACT methods the cancer is colorectal cancer or hepatocellular carcinoma. In certain embodiments of the described ACT methods, the composition to which the dendritic cells are exposed ex vivo comprises or consists of or consists essentially of tricin 7-O-β-D-glucopyranoside, and/or acylated tricin 7-O-β-D-glucopyranoside (6a).

According to certain aspects of the invention, a method to treat colorectal cancer by administering AdIL-12 and tricin 7-O-β-D-glucopyranoside to a subject having at least one colorectal tumor is provided. AdIL-12 is administered intratumorally.

According to certain aspects of the invention a method is provided to eradicate a colorectal tumor, by administering AdIL-12 (administered intratumorally), and tricin 7-O-β-D-glucopyranoside.

According to certain aspects of the invention an anticancer composition for treating colorectal tumors comprising AdIL-12 and tricin 7-O-β-D-glucopyranoside is provided. The composition may be effective in preventing or inhibiting growth of a colorectal tumor as well as in in eradication of colorectal tumors.

Thus, provided herein are methods of reducing the proliferation of cancer cells, reducing the size of one or more tumors, eradicating one or more tumors, reducing a risk of mortality due to cancer, or extending a life of a subject in need thereof, which comprises administering to the subject in need thereof a pharmaceutically effective amount of tricin 7-O-β-D-glucopyranoside, or its derivatives in combination with AdIL-12.

### BRIEF DESCRIPTION OF THE DRAWINGS

FIG. 1 depicts the chemical structure of tricin and tricin 7-O-β-D-glucopyranoside (Antartina) and related derivatives and analogues synthesized and tested as described herein (“Antartina 1a” corresponds to tricin 7-O-β-D-glucopyranoside).

FIGS. 2A, 2B, and 2C depict MTT assay absorbance data obtained from incubation of tricin 7-O-β-D-glucopyranoside (referred to as Antartina) and related derivatives with HEPA129 cells at various time points, including: 24 hrs (FIG. 2A), 48 hrs (FIG. 2B), and 72 hrs (FIG. 2C).

FIGS. 3A, 3B, and 3C depict MTT assay absorbance data obtained from incubation of tricin 7-O-β-D-glucopyranoside (referred to as Antartina) and related derivatives with CT26 cells at various time points, including: 24 hrs (FIG. 3A), 48 hrs (FIG. 3B), and 72 hrs (FIG. 3C).

FIG. 4 depicts cell size (mm³) in mice of CT26 tumors after CT26 cell line injection into mice. From left to right, first panel are the results of a control containing only carrier, second panel is treatment of the mouse with tricin, third panel shows the results of treatment of the mouse with tricin 7-O-β-D-glucopyranoside (referred to as Antartina®), and the last panel on the right shows treatment of the mouse with acetylated tricin 7-O-0-β-glucopyranoside (referred to as Antartina acetilada). The bottom panel provides a summary of all results in one graph.

FIG. 5A shows tumor volume vs. treatment for C3H/He mice that were injected with 200 mg/kg of thioacetamide (3 times a week for 6 weeks). On day 28, mice were anesthetized and orthotopic tumors were established by sub-capsular inoculation of 1.25×10⁵ Hepa129 cells into the left liver lobe by laparotomy (day 0). Five days after tumor implantation, mice were distributed in groups (n=6-8/group) and received: i) saline (control, FIG. 5A, left panel); ii) acetylated tricin 7-O-β-D-glucopyranoside (referred to as Acetilada) (50 mg/kg, 3 times per week, FIG. 5A, right panel). Fifteen days after tumor implantation, mice were sacrificed and tumor volume was quantified, as depicted in FIG. 5B (control, left panel; acetylated tricin 7-O-β-D-glucopyranoside (referred to as Acetilada), right panel).

FIG. 6 shows effect of tricin 7-O-β-D-glucopyranoside (referred to as Antartina) and IL-12 gene therapy in combination on a murine model of CRC (CT26 cells) established in BALB/c mice. Mouse CT26 cells (5×10⁵) were subcutaneously inoculated on day 0. On day 7, when tumors reached a diameter of 5-7 mm, animals were divided into 4 groups: saline, tricin 7-O-β-D-glucopyranoside (referred to as Antartina) (Antartina 50 mg/kg/ip 3 times/week), AdIL12 (intratumorally 109 TCI 50) at day 15, and the combination of tricin 7-O-β-D-glucopyranoside (Antartina) (Antartina 50 mg/kg/ip 3 times/week), and AdIL-12 (intratumorally 109 TCI 50) at day 15. Tumor volume is shown in the graph on the left panel vs. days after CT26 inoculation. Excised tumors are depicted in the right panel. Tumor volume was measured by caliper 2-times a week. Complete tumor regressions were confirmed macroscopically at day 21.

### DETAILED DESCRIPTION

In colorectal carcinoma (CRC) patients, distant metastatic disease is present at initial diagnosis in nearly 25% of patients. The majority of patients with metastatic CRC have an incurable disease; therefore, new therapies are needed to address this cancer. Agents derived from medicinal plants have already demonstrated therapeutic activities in human cancer cells. Tricin 7-O-β-D-glucopyranoside (Antartina) is an antitumor agent isolated from Deschampsia antarctica Desv. (Malvicini et al., Mol. Cancer Therap., 2018, 17(5):966-976). In that study the aim was to evaluate the antitumor properties of tricin 7-O-β-D-glucopyranoside (Antartina) in CRC models. Human and murine CRC cell lines were employed to investigate proliferation, apoptosis, and cell cycle effects of Antartina therapy in vitro. immune response of Antartina against cancer cells in the CRC model was also investigated using a cytotoxic T lymphocyte (CTL) assay, analyzing dendritic cell activation and intratumor T cell sub-population, and by tumor re-challenge experiments.

Here, it is shown that tricin 7-O-β-D-glucopyranoside (Antartina) and its derivatives, such as acetylated tricin 7-O-β-D-glucopyranoside, in combination with immune-gene therapy using an adenovirus encoding IL-12, induces a potent antitumoral response against CRC in mice when combined with AdIL-12. It is also disclosed that acetylated tricin 7-O-β-D-glucopyranoside has a superior antitumoral effect in comparison with native tricin 7-O-β-D-glucopyranoside (Antartina) in mice with CRC. It is shown here also, that tricin 7-O-β-D-glucopyranoside (Antartina) in combination with immuno-gene therapy (adenovirus-IL12) shows a synergistic antitumoral effect 80% remission of the tumors. Additionally, analogues or derivatives of tricin-7-O-β-D-glucopyranoside are shown to have a potent effect on hepatocarcinoma cells.

Tricin 7-O-β-D-glucopyranoside inhibits in vitro human CRC cell proliferation. In an immunocompetent CRC mouse model, tricin 7-O-β-D-glucopyranoside potently inhibited tumor growth and liver metastases, leading to complete tumor regressions in >30% of mice, along with an increased animal survival rate. In addition, tricin 7-O-β-D-glucopyranoside induced a potent specific cytotoxic T cell response against CRC and a long lasting antitumor immunity. Interestingly, tricin 7-O-β-D-glucopyranoside_increased tumor immunogenicity and stimulated dendritic cell activation. No toxic effects were observed at the doses employed. These findings show that tricin 7-O-β-D-glucopyranoside_has the ability to induce antitumor immunity against CRC and is useful for the treatment of CRC.

While various aspects and embodiments have been illustrated and described in detail, above, such illustration and description are to be considered illustrative or exemplary and not restrictive. It will be understood that changes and modifications can be made by those of ordinary skill within the scope and spirit of the following claims. In particular, this description is intended to encompass further embodiments with any combination of features from different embodiments described above and below.

The compositions and methods described herein are additionally described by way of the following illustrative non-limiting examples that provide a better understanding of these embodiments and their many advantages. The following examples are included to demonstrate certain useful embodiments. It should be appreciated by those of skill in the art that the techniques disclosed in the examples which follow represent compositions and methods that function well, and thus can be considered to constitute preferred embodiments. However, those of skill in the art should, in light of the present disclosure, appreciate that many changes can be made in the specific embodiments that are disclosed and still obtain a like or similar result without departing from the spirit and scope of this disclosure.

### EXAMPLES

Melting points were determined in open capillary tubes with a Stuart Scientific SMP3 melting point apparatus. Infrared (IR) spectra were obtained with Fourier-transform infrared (FTIR) Cary 630 (Agilent Technologies, Santa Clara, CA, U.S.) with diamond attenuated total reflectance (ATR) spectrophotometer. Nuclear magnetic resonance (NMR) ¹H and ¹³C spectra were recorded using Varian Gemini 200, Varian Unity 300/500 MHz, or Varian Mercury 300/400 systems at room temperature. (Varian Medical Systems, Palo Alto, CA, U.S.). Chemical shifts are given in ppm (δ) downfield from tetramethylsilane (TMS). Coupling constants (J) are in Hertz [Hz] and signals are described as follows: s, singlet; d, doublet; t, triplet; q, quadruplet; m, multiplet; br, broad; app, apparent. Analysis of the NMR free induction decays (FIDs) were performed using Mestrenova 6.0.2 software (Mestrelab Research, Santiago de Compostela, Spain). Low resolution mass spectra were obtained on Agilent Technologies 6120 Quadrupole Liquid Chromatography-Mass Spectrometry (LC-MS) in electrospray ionization (ESI) with an Agilent Technologies 1260 Infinity LC system using SeQuant Zic-Hilic (Merck-Millipore) 150 mm×4.6 mm, 5 μm column (Agilent Technologies, Santa Clara, CA, U.S.). Agilent formula predictor software was used to process the data. MS was reported in units of mass to charge ratio (m/z). The analysis was performed with a flow rate of 1 mL/min maintaining the column temperature at 25° C. The detector was set at 214-254 nm±16 nm. Mass Detection: Scan 50-1000 m/z. The mobile phase consisted of phase A (200 mM ammonium acetate, pH 5.2, without ethylenediaminetetraacetic acid (EDTA)) and phase B (acetonitrile 80% and solution of ammonium acetate 20%), isocratic method. High-resolution mass spectrometry analysis (time-of-flight, TOF) was performed using an Agilent 6210 time-of-flight LC/MS. (Agilent Technologies, Santa Clara, CA, U.S.). All chemicals were obtained from commercial sources and used as received without extra purification. Palladium on carbon 10 wt % loading matrix activated carbon support (Pd (C), 205699), ammonia solution 32% (105426) were purchased from Sigma-Aldrich, St. Louis, MO, U.S. Thin layer chromatography (TLC) analyses were performed using silica gel (Kieselgel 60 7254, Macherey-Nagel, Duren, Germany) and the spots were visualized under ultraviolet (UV) light. The column chromatography was performed with silica gel 60 (40-63 μm, Merck & Co., Kenilworth, NJ, U.S.) columns, using the eluent reported in each case.

Synthesis of tricin 7-O-β-D-glucopyranoside (Antartina) was previously described in U.S. Pat. No. 10,058,582 (the entire contents of which are incorporated herein by reference, especially those sections outlining the synthesis of tricin 7-O-β-D-glucopyranoside.

Adenovirus IL-12 constructs are as previously reported. (Mazzolini et al., Cancer Gene Therapy, 6(6):514-522, 1999).

Tricin 7-O-β-D-glucopyranoside_was detected and obtained by isolation using semi-preparative high performance liquid chromatography (HPLC) from aqueous extracts of Deschampsia antarctica Desv. (Poaceae). The purity and structural identification of the isolated peak 10 was performed by Nuclear Magnetic Resonance (NMR) and analyzed by mass spectrometry (MS). Peak 10 isolated from Deschampia antartica extract, is tricin 7-O-P-D-glucopyranoside, also called 5-hydroxy-2-(4-hydroxy-3,5-dimethoxy-phenyl)-7-[(2S,3S,4R,5S,6R)-3,4,5-trihydroxy-6-(hydroxymethyl) tetrahydropyran-2-yl] oxychromen-4-one.

Mouse CT26 tumor cell line, an undifferentiated murine colorectal carcinoma (CRC) cell line established from N-nitroso-N-methylurethan-induced transplantable tumor in BALB/c (H-2d) mice, BNL cells (a hepatoma cell line) were used in the studies below and can be obtained from the University of Navarra, Spain. Human cell lines were from American Type Culture Collection (ATCC, Manassas, VA, U.S.), including LoVo (ATCC-CCL229), ASG (ATCC-CCRL-79) and lung fibroblast Wi38 (ATCC-CCL75). All lines were tested for mycoplasma and cultured in complete DMEM (GIBCO, Themo Fisher Scientific, mM glutamine, 100 U/mL penicillin, 100 mg/mL streptomycin, and 10% heat inactivated fetal bovine serum (FBS, GIBCO, Thermo Fisher Scientific) and incubated 37° C. in 5% CO₂.

All animals were from Envigo (Barcelona, Spain) and were maintained at the Spanish National Cancer Research Center (CNIO) Animal Facility (AAALAC accredited) in accordance with guidelines stated in the International Guiding Principles for Biomedical Research Involving Animals (CIOMS). All animal experiments were approved by the Competent Authority of Communidad de Madrid (project PROEX 104/16).

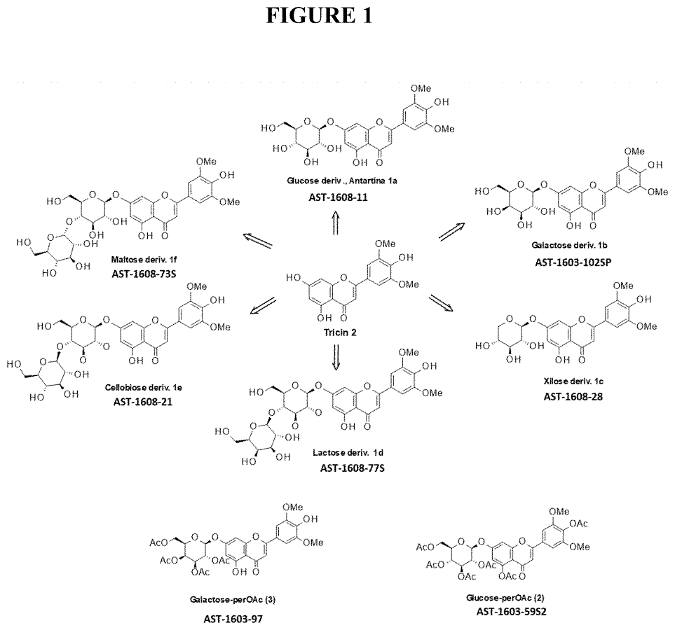

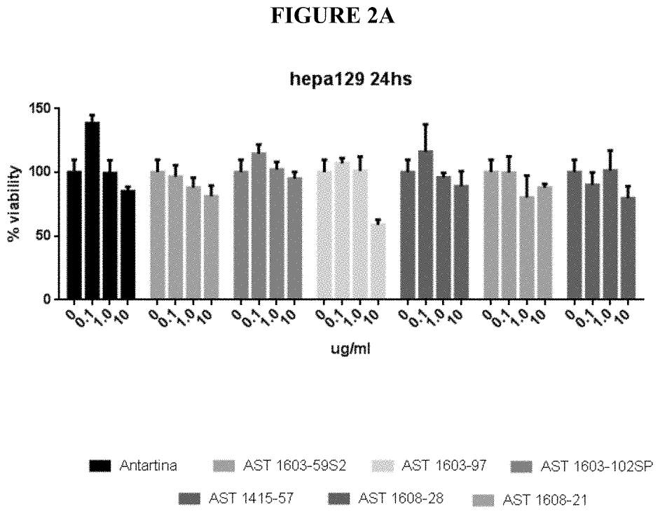

### Example 1: Synthesis of Tricin 7-O-β-D-glucopyranoside-derivative, Per Acetylated (AST-1603-59S2, 6a, FIG. 1)

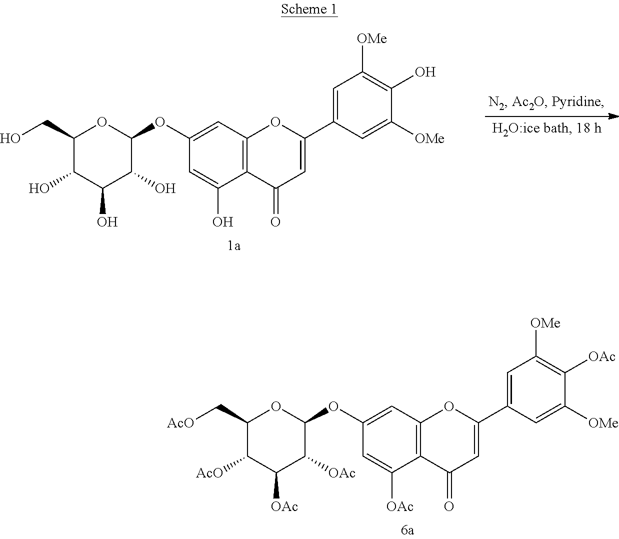 [SMILES: COc1cc(-c2cc(=O)c3c(O)cc(O[C@@H]4O[C@H](CO)[C@@H](O)[C@H](O)[C@H]4O)cc3o2)cc(OC)c1O ; COc1cc(-c2cc(=O)c3c(OC(C)=O)cc(O[C@@H]4O[C@H](COC(C)=O)[C@@H](OC(C)=O)[C@H](OC(C)=O)[C@H]4OC(C)=O)cc3o2)cc(OC)c1OC(C)=O]

A mixture of Tricin 7-O-β-D-glucopyranoside 1a (0.5 g, 1.02 mmol, 1.0 eq.) and pyridine (14 mL, 173.4 mmol) in a water:ice bath (4° C.) was treated with Ac₂O (14 mL) dropwise. The reaction mixture is warmed up at room temperature and stirred overnight. After that, the solvents were removed under reduced pressure and the residue was treated with methyl-t-butyl ether (MTBE) (50 mL) to obtain 0.5 g (56%) of 6a as a white solid. M. p.: 239.1-241.8° C. 1H-NMR (CDC13, 500 MHz)□(ppm): 2.05 (m, 6H), 2.06 (s, 3H), 2.08 (s, 3H), 2.37 (s, 3H), 2.44 (s, 3H), 3.91 (s, 6H), 3.99 (br s, 1H), 4.20 (d, J=11.8 Hz, 1H), 4.28 (dd, J=12.6 Hz, J=5.6 Hz, 1H), 5.17 (t, J=9.0 Hz, 1H), 5.36-5.23 (m, 3H), 6.56 (s, 1H), 6.70 (s, 1H), 7.02 (s, 1H), 7.05 (s, 2H). 13C-RMN (CDC13, 125 MHz)□(ppm): 178.8, 173.9, 172.7, 172.3, 172.0, 171.9, 170.9, 164.4, 162.5, 160.8, 155.3, 153.3, 134.2, 131.9, 115.4, 111.6, 111.4, 105.7, 105.6, 100.5, 75.1, 74.8, 73.5, 70.7, 64.6, 59.0, 23.7, 23.2, 23.0. HPLC-UV-MS: tR=15.96 min, Purity (at 254 nm): 98%; MS (ESI, positive mode): m/z=745.2, [M+H]+; MS (ESI, negative mode): m/z=743.1 (M−H)−. HRMS calculated for C₃₅H₃₆O₁₈, 744.1902 [M+H]+, found, 744.1902.

### Example 2: Synthesis of tricin 7-O-β-D-glucopyranoside Derivative [3,4,5-triacetoxy-6-[5-hydroxy-2-(4-hydroxy-3,5-dimethoxy-phenyl)-4-oxo-chromen-7-yl]oxy-tetrahydropyran-2-yl]methyl acetate (AST-1603-097, 5b, FIG. 1)

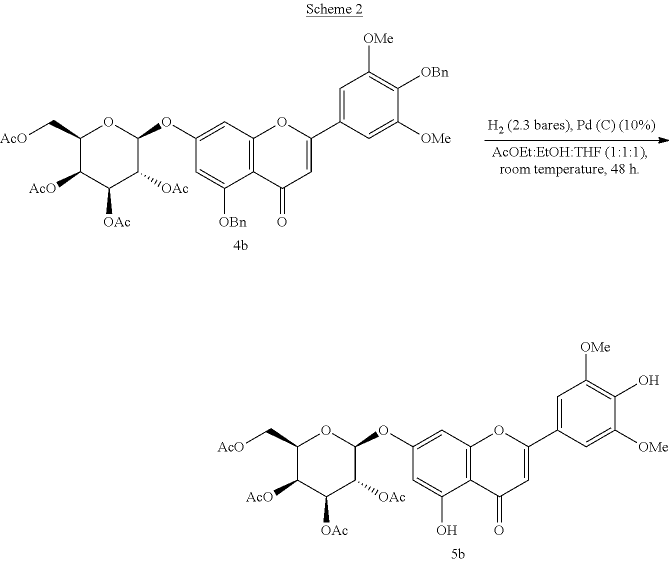 [SMILES: COc1cc(-c2cc(=O)c3c(OCc4ccccc4)cc(O[C@@H]4O[C@H](COC(C)=O)[C@H](OC(C)=O)[C@H](OC(C)=O)[C@H]4OC(C)=O)cc3o2)cc(OC)c1OCc1ccccc1 ; COc1cc(-c2cc(=O)c3c(O)cc(O[C@@H]4O[C@H](COC(C)=O)[C@H](OC(C)=O)[C@H](OC(C)=O)[C@H]4OC(C)=O)cc3o2)cc(OC)c1O]

A solution of 4b (6.0 g; 7.14 mmol, 1.0 eq.) in AcOEt:EtOH:THF (1:1:1, 250 mL, 1 g/42 mL) was treated with Pd (C) (10%) (10% w/w, 0.6 g). The reaction mixture was purged with N₂ (3×2 bars) and H₂ (3×2 bars). Finally, the reaction was treated with H₂ (2.3 bars) at room temperature for 48 hours. After that, the reaction mixture was filtered through celite and the solvents were removed under reduced pressure. The residue was treated with Et₂O (50 mL) to obtain 3.50 g (75%) of 3 as a brown-red solid. M. p.: 117.2-121.8° C. 1H-NMR (DMSO-d6, 500 MHz)□(ppm): 1.95 (s, 3H), 2.01 (s, 3H), 2.05 (s, 3H), 2.15 (s, 3H), 3.88 (s, 6H), 4.12 (br s, 2H), 4.54 (br s, 1H), 5.24 (m, 2H), 5.35 (br s, 1H), 5.68 (br s, 1H), 6.47 (s, 1H), 6.85 (s, 1H), 7.39 (s, 1H), 7.36 (s, 2H), 9.39 (br s, 1H), 13.04 (br s, 1H); 13C-RMN (DMSO-d6, 125 MHz)□(ppm): 185.2, 173.0, 172.9, 172.6, 172.3, 167.5, 164.7, 164.5, 159.9, 154.9, 151.3 (2C), 143.3, 142.2, 131.0, 127.9, 123.2, 108.9, 107.7 (2C), 106.9, 101.9, 99.9, 98.6, 73.8, 73.3, 71.1, 70.3, 64.6, 59.6, 33.5. HPLC-UV-MS: tR=15.67 min, Purity (at 254 nm): 98%; MS (ESI, positive mode): m/z=661, [M+H]+; MS (ESI, negative mode): m/z =659.0 (M−H)−. HRMS calculated for C₃₁H₃₃O₁₆, 661.1763 [M+H]+, found, 661.1731.

### Example 3: Synthesis of tricin 7-O-β-D-glucopyranoside_derivative 5-hydroxy-2-(4-hydroxy-3,5-dimethoxy-phenyl)-7-[3,4,5-trihydroxy -6-(hydroxymethymethyl)tetrahyropyran-2-yl]oxy -chromen-4-one (AST-1603-102 SP, 1b, FIG. 1)

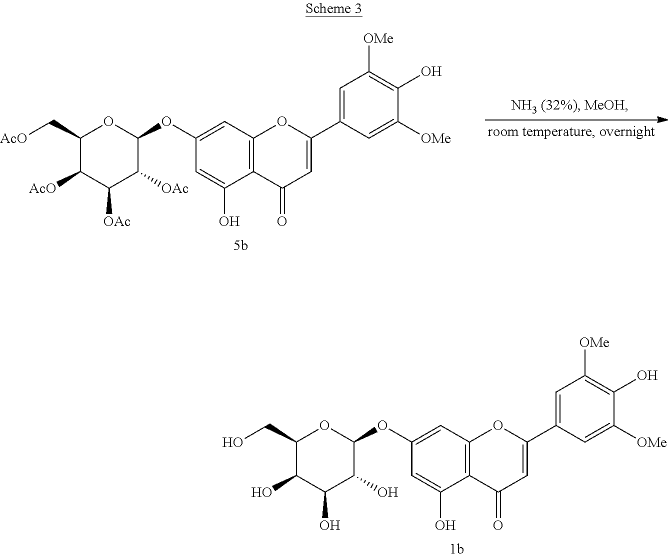 [SMILES: COc1cc(-c2cc(=O)c3c(O)cc(O[C@@H]4O[C@H](COC(C)=O)[C@H](OC(C)=O)[C@H](OC(C)=O)[C@H]4OC(C)=O)cc3o2)cc(OC)c1O ; COc1cc(-c2cc(=O)c3c(O)cc(O[C@@H]4O[C@H](CO)[C@H](O)[C@H](O)[C@H]4O)cc3o2)cc(OC)c1O]

[3,4,5-triacetoxy-6-[5-hydroxy-2-(4-hydroxy-3,5-dimethoxy-phenyl)-4-oxo-chromen-7-yl]oxy-tetrahydropyran-2-yl]methyl acetate (AST-1603-097, 5b, 2.0 g, 3.03 mmol, 1.0 eq.) was dissolved in MeOH (140 mL; 1 g/70 mL) and finally treated with 32% aqueous NH₃ solution (62 mL). The reaction mixture was stirred at room temperature overnight. After that, the solvents were removed under reduced pressure. The residue was treated with DMSO (1 mL) and distilled H₂O (10 mL). The solid was removed after filtration isolating 1.0 g (67%) of 1b as a brown solid. M. p.: 260.2-263.3° C. 1H-NMR (DMSO-d6, 500 MHz)□(ppm): 3.50-3.62 (m, 4H), 3.69 (dt, J=14.0 Hz, J=7.9 Hz, 2H), 3.87 (s, 6H), 4.54 (d, J=4.6 Hz, 1H), 4.71 (t, J=5.5 Hz, 1H), 4.89 (d, J=5.8 Hz, 1H), 4.99 (d, J=7.7 Hz, 1H), 5.22 (d, J=5.2 Hz, 1H), 6.45 (d, J=2.2 Hz, 1H), 6.92 (d, J=2.2 Hz, 1H), 7.06 (s, 1H), 7.35 (s, 2H), 9.36 (br s, 1H), 12.9 (br s, 1H). 13C-RMN (DMSO-d6, 125 MHz)□(ppm): 185.3, 167.3, 166.3, 163.9, 159.9, 151.3 (2C), 142.9, 123.2, 108.5, 107.4 (2C), 106.9, 104.1, 102.6, 98.1, 78.9, 76.3, 73.2, 71.3, 63.6, 59.5 (2C). HPLC-UV-MS: tR=10.09 min; Purity (at 254 nm): 98.5%; MS (ESI, positive mode): m/z=493, [M+H]+; MS (ESI, negative mode): m/z=491 (M−H)−.

HRMS calculated for C23H25O₁₂, 493.1340 [M+H]+.

### Example 4: Synthesis of tricin 7-O-β-D-glucopyranoside Xylose Derivative 1c (AST-1608-28, 1c, FIG. 1)

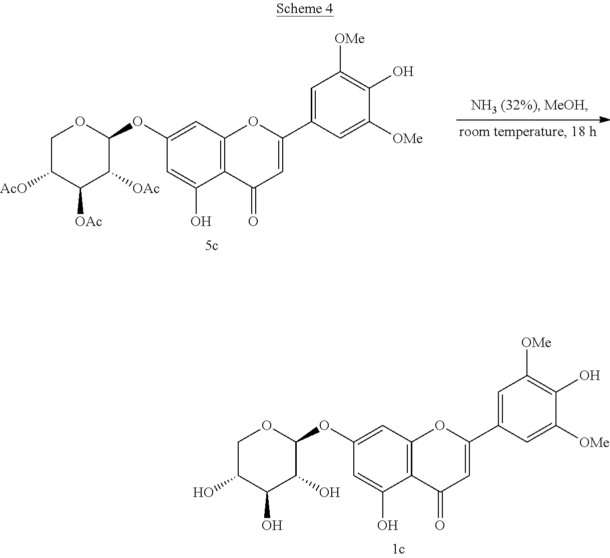 [SMILES: COc1cc(-c2cc(=O)c3c(O)cc(O[C@@H]4OC[C@@H](OC(C)=O)[C@H](OC(C)=O)[C@H]4OC(C)=O)cc3o2)cc(OC)c1O ; COc1cc(-c2cc(=O)c3c(O)cc(O[C@@H]4OC[C@@H](O)[C@H](O)[C@H]4O)cc3o2)cc(OC)c1O]

The xylose derivative precursor, (AST1608-1353) 5c, (2.9 g, 4.9 mmol, 1.0 eq.) was dissolved with MeOH (200 mL, 1 g/70 mL) and treated with 32% aqueous NH₃ solution (100 mL). The reaction mixture was stirred at room temperature overnight. After that, the solvents were removed under reduced pressure. The residue was treated with DMSO (2 mL) and distilled H₂O (40 mL). After precipitation, the solid was collected after filtration yielding 1.5 g (66%) of 1c as a pale yellow-brown solid. M. p.: 245.3-249.8° C. 1H-NMR (DMSO-d6, 500 MHz)□(ppm): 3.19-3.46 (m, 5H), 3.76 (d, J=5.8 Hz, 1H), 3.88 (s, 6H), 5.08 (m, 2H), 5.42 (br s, 1H), 6.42 (d, J=1.6 Hz, 1H), 6.88 (d, J=1.6 Hz, 1H), 7.05 (s, 1H), 7.36 (s, 2H), 9.26 (br s, 2H), 12.90 (br s, 1H). 13C-RMN (DMSO-d6, 125 MHz)□(ppm): 185.2, 167.3, 165.7, 164.2, 160.0, 151.2 (2C), 143.2, 123.2, 108.5, 107.7 (2C), 106.8, 103.4, 102.5, 98.1, 79.3, 75.9, 72.3, 68.9, 59.5 (2C). HPLC-UV-MS: tR=11.9 min; Purity (at 254 nm): 98.1%; MS (ESI, positive mode): m/z=463.0 [M+H]+, 485 [M+Na]+; MS (ESI, negative mode); m/z=461.0 (M−H)−. HRMS calculated for C₂₂H₂₃O₁₁, 463.1234 [M+H]+, found, 463.1208.

### Example 5: Synthesis of tricin 7-O-β-D-glucopyranoside derivative 7-[(3R,4R,5S,6R)-3,4-dihydroxy-6-(hydroxymethyl)-5-[(3R,4S,5R,6R)-3,4,5-trihydroxy-6-(hydroxymethyl)tetrahydropyran-2-yl]oxy-tetrahydropyran-2-yl]oxy-5-hydroxy-2-(4-hydroxy-3,5-dimethoxy-phenyl)chromen-4-one (AST-1608-77s, 1d, FIG. 1)

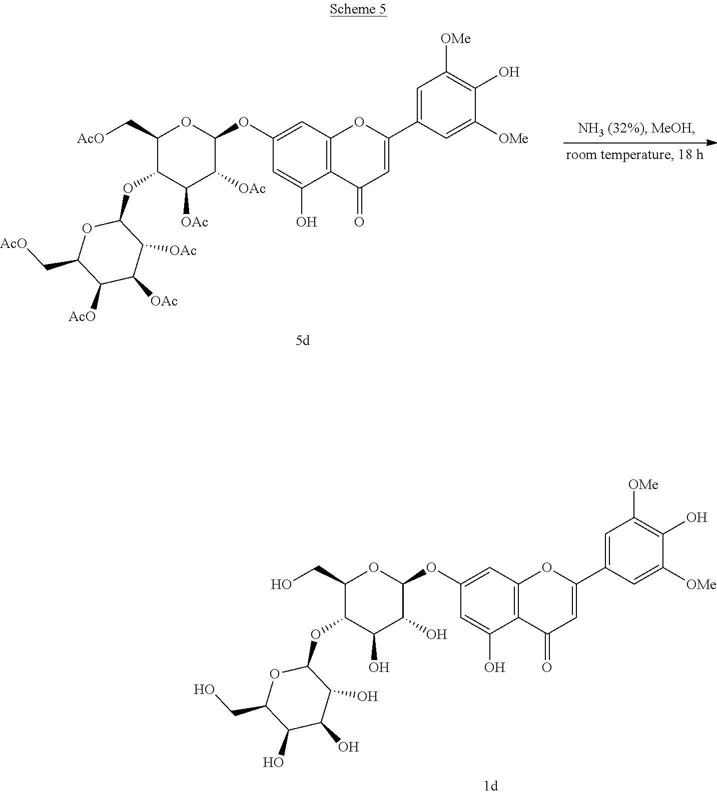 [SMILES: COc1cc(-c2cc(=O)c3c(O)cc(O[C@@H]4O[C@H](COC(C)=O)[C@@H](O[C@@H]5O[C@H](COC(C)=O)[C@H](OC(C)=O)[C@H](OC(C)=O)[C@H]5OC(C)=O)[C@H](OC(C)=O)[C@H]4OC(C)=O)cc3o2)cc(OC)c1O ; COc1cc(-c2cc(=O)c3c(O)cc(O[C@@H]4O[C@H](CO)[C@@H](O[C@@H]5O[C@H](CO)[C@H](O)[C@H](O)[C@H]5O)[C@H](O)[C@H]4O)cc3o2)cc(OC)c1O]

[(2R,3S,4R,5R)-5-acetonyl-4-acetoxy-6-[5-hydroxy-2-(4-hydroxy-3,5-dimethoxy-phenyl)-4-oxo-chromen-7-yl]oxy-3-[(3R,4S,5S,6R)-3,4,5-triacetoxy-6-(acetoxy methyl)tetrahydropyran-2-yl]oxy-tetrahydropyran-2-yl]methyl acetate (AST-1608-69) 5d (4.0 g, 4.22 mmol, 1.0 eq.) was dissolved with MeOH (280 mL, 1 g/70 mL) and finally treated with 32% aqueous NH₃ solution (104 mL). The reaction mixture was stirred overnight at room temperature. After that, the solvents were removed under reduced pressure. The residue was treated with DMSO (2 mL) and distilled H₂O (20 mL). The solid was removed after filtration isolating 1.6 g (58%) of 1d as a pale yellow-brown solid. M. p.: 275.4-279.2° C. 1H -NMR (DMSO-d6, 500 MHz)□(ppm): 3.38 (dd, J=15.7 Hz, J=6.4 Hz, 2H), 3.44 -3.57 (m, 5H), 3.63 (m, 4H), 3.80 (dd, J=10.3 Hz, J=5.2 Hz, 1H), 3.88 (s, 6H), 4.23 (d, J=7.2 Hz, 1H), 4.52 (d, J=4.6 Hz, 1H) 4.67 (dd, J=12.6 Hz, J=5.7 Hz, 2H), 4.78 (d, J=5.1 Hz, 1H), 4.83 (d, J=1.3 Hz, 1H), 5.08 (d, J=4.5 Hz, 1H), 5.15 (d, J=7.8 Hz, 1H), 5.55 (d, J=5.3 Hz, 1H), 6.47 (d, J=2.0 Hz, 1H), 6.93 (d, J=2.0 Hz, 1H), 7.07 (s, 1H), 7.35 (s, 1H), 9.36 (br s, 2H), 12.9 (br s, 1H). 13C-RMN (DMSO-d6, 125 MHz)□(ppm): 185.0, 167.2, 165.9, 164.1, 159.9, 151.3 (2C), 143.1, 123.2, 108.5, 107.6 (2C), 106.7, 102.5, 98.3, 83.1, 78.7, 78.3, 77.8, 76.4, 75.9, 73.7, 71.3, 63.5, 63.0, 59.6 (2C). HPLC-UV-MS: tR=10.06 min; Purity (at 254 nm): 95.4%; MS (ESI, positive mode): m/z=655 [M+H]+; MS (ESI, negative mode); m/z=653.3 (M−H)−. HRMS calculated for C₂₉H₃₅O₁₇, 665.1868 [M+H]+, found, 655.1835.

### Example 6: Synthesis of tricin 7-O-β-D-glucopyranoside derivative 7-[(3R,4R,5S,6R)-3,4-dihydroxy-6-(hydroxymethyl)-5-[(3R,4S,5S,6R)-3,4,5-trihydroxy-6-(hydroxymethyl)tetrahydropyran-2-yl]oxy-tetrahydropyran-2-yl]oxy-5-hydroxy-2-(4-hydroxy-3,5-dimethoxy-phenyl)chromen-4-one (AST-1608-21, 1e, FIG. 1)

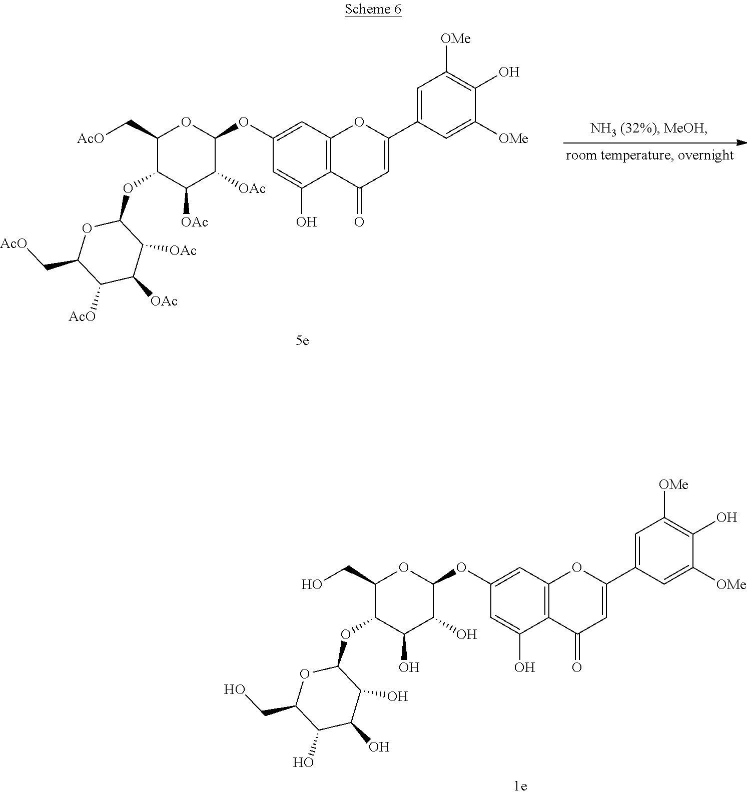 [SMILES: COc1cc(-c2cc(=O)c3c(O)cc(O[C@@H]4O[C@H](COC(C)=O)[C@@H](O[C@@H]5O[C@H](COC(C)=O)[C@@H](OC(C)=O)[C@H](OC(C)=O)[C@H]5OC(C)=O)[C@H](OC(C)=O)[C@H]4OC(C)=O)cc3o2)cc(OC)c1O ; COc1cc(-c2cc(=O)c3c(O)cc(O[C@@H]4O[C@H](CO)[C@@H](O[C@@H]5O[C@H](CO)[C@@H](O)[C@H](O)[C@H]5O)[C@H](O)[C@H]4O)cc3o2)cc(OC)c1O]

[(2R,3S,4R,5R)-5-acetonyl-4-acetoxy-6-[5-hydroxy-2-(4-hydroxy-3,5-dimethoxy-phenyl)-4-oxo-chromen-7-yl]oxy-3-[(3R,4S,5R,6R)-3,4,5 -tri acetoxy-6-(acetoxy methyl)tetrahydropyran-2-yl]oxy-tetrahydropyran-2-yl]methyl acetate (AST-1608-16) 5e (3.89 g, 4.1 mmol, 1.0 eq.) was dissolved with MeOH (280 mL, 1 g/72 mL) and finally treated with 32% aqueous NH₃ solution (104 mL). The reaction mixture was stirred at room temperature overnight. After that, the solvents were removed under reduced pressure. The residue was treated with DMSO (3 mL) and distilled H₂O (30 mL). The solid was collected after filtration, isolating 1.7 g (64%) of 1e as a pale yellow-brown solid. M. p.: 243.2-246.5° C. 1H-NMR (DMSO-d6, 500 MHz)□(ppm): 2.96-3.10 (m, 2H), 3.19 (m, 3H), 3.40 (dd, J =19.9 Hz, J=11.9 Hz, 2H), 3.47 (dt, J=12.0 Hz, J=9.5 Hz, 1H), 3.64 (m, 2H), 3.67-3.74 (m, 2H), 3.79 (dd, J=10.2 Hz, J=4.7 Hz, 1H), 3.87 (s, 6H), 4.28 (d, J=7.9 Hz, 1H), 4.60 (t, J=5.2 Hz, 1H), 4.69 (t, J=5.6 Hz, 1H), 4.82 (d, J=1.9 Hz, 1H), 5.06-4.94 (m, 2H), 5.14 (d, J=7.8 Hz, 1H), 5.22 (d, J=4.7 Hz, 1H), 5.54 (d, J=5.1 Hz, 1H), 6.46 (d, J=2.1 Hz, 1H), 6.92 (d, J=2.1 Hz, 1H), 6.46 (d, J=2.1 Hz, 1H), 6.92 (d, J=2.1 Hz, 1H), 7.06 (s, 1H), 7.35 (s, 1H), 9.33 (br s, 2H), 12.9 (br s, 1H). 13C-RMN (DMSO-d6, 125 MHz)□(ppm): 185.1, 167.2, 165.9, 164.2, 159.9, 151.3 (2C), 143.1, 123.0, 108.5, 107.6 (2C), 106.9, 106.2, 102.7, 102.5, 98.3, 82.9, 79.8, 79.5, 78.4, 77.9, 76.4, 75.9, 73.1, 64.2, 63.0, 59.4 (2C). HPLC-UV-MS: tR=10.20 min; Purity (at 254 nm): 100%; MS (ESI, positive mode): m/z=655 [M+H]+, 677 [M+Na]+; MS (ESI, negative mode): m/z=653.3 (M+H)+. HRMS calculate d for C₂₉H₃₅O₁₇, 665.1868 [M+H]+, found, 665.1833.

### Example 7: Synthesis of tricin 7-O-β-D-glucopyranoside_derivative 7-[(2S,3R,4R,5S,6R) -3,4-dihydroxy-6-(hydroxymethyl)-5-[(2S,3R,4S,5S,6R)-3,4,5-trihydroxy-6-(hydroxymethyl)tetrahydropyran-2-yl]oxy-tetrahydropyran-2-yl]oxy-5-hydroxy-2-(4-hydroxy-3,5-dimethoxy-phenyl)chromen-4-one (AST-1608-73s, 1f, FIG. 1)

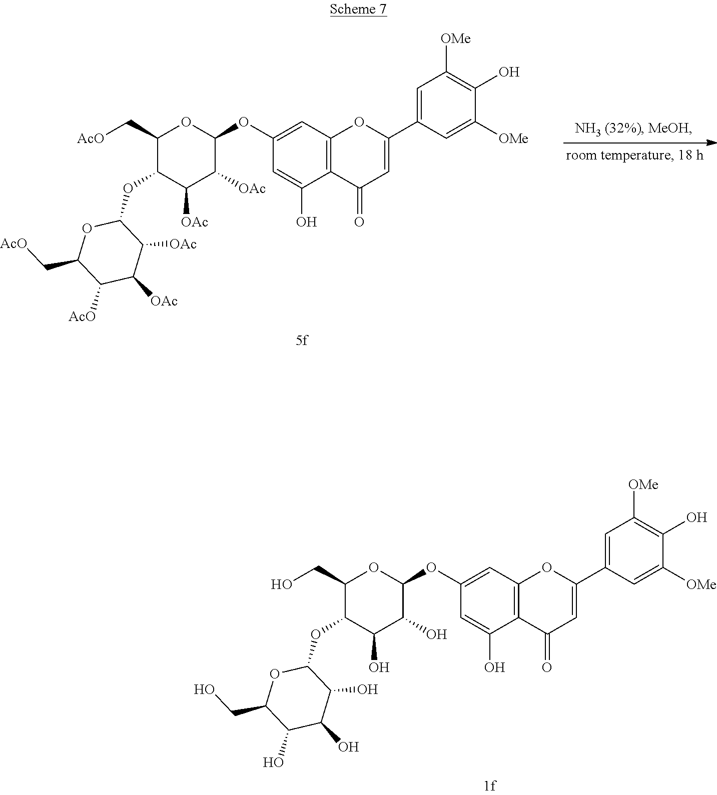 [SMILES: COc1cc(-c2cc(=O)c3c(O)cc(O[C@@H]4O[C@H](COC(C)=O)[C@@H](O[C@H]5O[C@H](COC(C)=O)[C@@H](OC(C)=O)[C@H](OC(C)=O)[C@H]5OC(C)=O)[C@H](OC(C)=O)[C@H]4OC(C)=O)cc3o2)cc(OC)c1O ; COc1cc(-c2cc(=O)c3c(O)cc(O[C@@H]4O[C@H](CO)[C@@H](O[C@H]5O[C@H](CO)[C@@H](O)[C@H](O)[C@H]5O)[C@H](O)[C@H]4O)cc3o2)cc(OC)c1O]

[(2R,3S,4R,5R,6S)-5-acetonyl-4-acetoxy-6-[5-hydroxy-2-(4-hydroxy-3,5-dimethoxy -phenyl)-4-oxo-chromen-7-yl]oxy-3-[(3R,4S,5R,6R)-3,4,5-triacetoxy-6-(acetoxymethyl)tetrahydropyran-2-yl]oxy-tetrahydropyran-2-yl]methyl acetate (AST1608-67) 5f (2.89 g, 2.95 mmol, 1.0 eq.) was dissolved with MeOH (202 mL, 1 g/70 mL) and finally treated with 32% aqueous NH₃ solution (75 mL). The reaction mixture was stirred at room temperature overnight. After that, the solvents were removed under reduced pressure.

The residue was treated with DMSO (2 mL) and distilled H₂O (20 mL). The solid was collected after filtration yielding 1.6 g (58%) of if as a pale yellow-brown solid. M. p.: 114.7-194.4° C. 1H-NMR (DMSO-d6, 500 MHz)□(ppm): 3.06 (t, J=8.1 Hz, 2H), 3.20-3.52 (m, 5H), 3.54-3.65 (m, 5H), 3.74 (d, J=10.9 Hz, 1H), 3.86 (s, 6H), 4.63 (br s, 3H), 4.91 (br s, 2H), 5.05 (d, J=3.6 Hz, 1H), 5.11 (d, J=7.7 Hz, 1H), 5.55 (br s, 4H), 6.44 (d, J=1.9 Hz, 1H), 6.90 (d, J=1.9 Hz, 1H), 7.03 (s, 1H), 7.31 (s, 2H). 13C-RMN (DMSO-d6, 125 MHz)□(ppm): 185.1, 167.2, 166.0, 164.2, 159.9, 151.3 (2C), 143.3, 123.2, 108.5, 107.6 (2C), 106.8, 103.8, 102.8, 102.6, 98.3, 82.0, 79.2, 78.6, 76.6, 76.4, 75.8, 75.5, 72.9, 63.9, 63.3, 59.4 (2C). HPLC-UV-MS: tR=10.09 min; Purity (at 254 nm): 96.1%; MS (ESI, positive mode): m/z=655 [M+H]+; MS (ESI, negative mode): m/z=653.3 (M+H)+. HRMS calculated for C₂₉H35O₁₇, 665.1868 [M+H]+, found, 655.1836.

### Example 8: Cell Viability Assays in HEPA129 and CT26 Cells

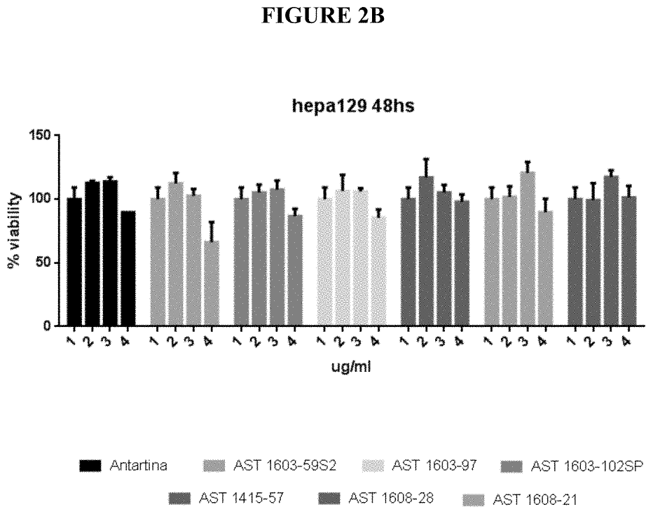

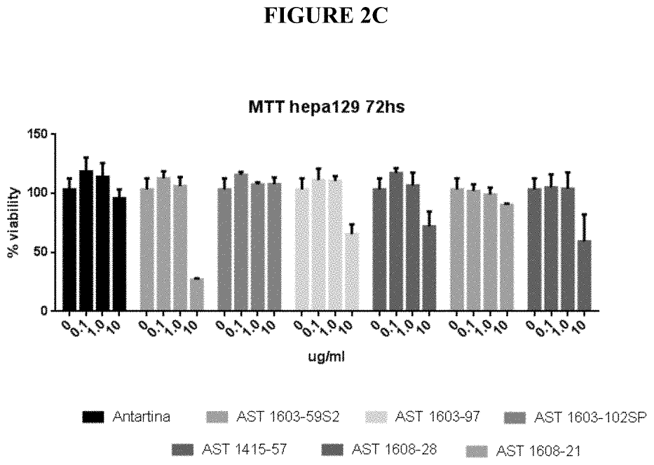

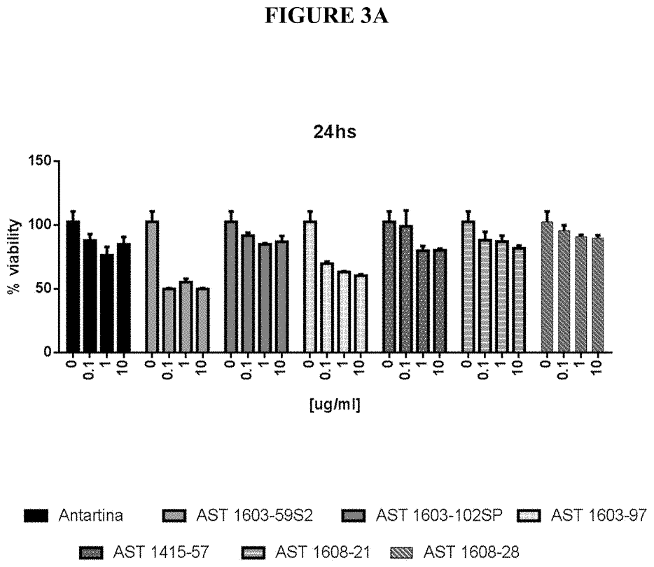

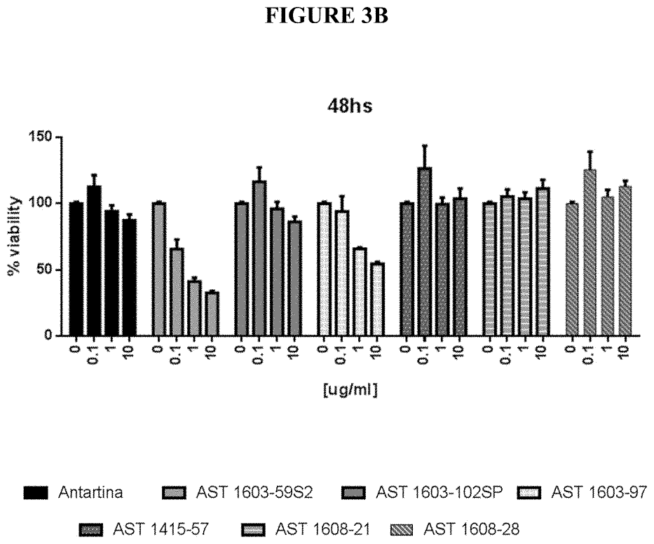

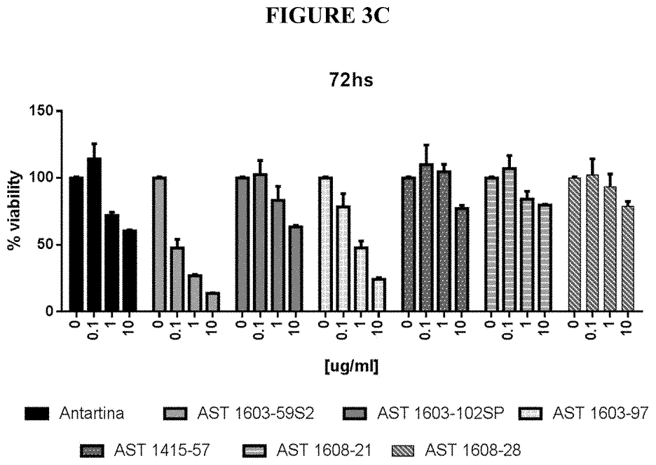

After synthesis of tricin 7-O-β-D-glucopyranoside and its derivatives, the molecules were tested for cytotoxicity in various cancer cell lines. For this, a cell viability test was performed. Briefly, gastric cancer cells, AGS, colon cancer, Lovo (human CRC cell line) , and as a control the Wi38 lung fibroblast line were employed. The cells were exposed to decreasing concentrations of molecules 1 and 3 from 1000 μM for 72 hrs. The negative control was just dimethyl sulfoxide (DMSO), the solvent in which the molecules to be tested were dissolved, with no active agent. Afterwards the cells were washed, and the MTS (tetrazolium dye) colorimetric compound was added for the MTT assay. It was determined that molecules 1 and 3 were able to inhibit approximately in 50% the cell viability of the AGS and Lₒᵥₒ cancer lines at a concentration of 1000 and 500 μM, as shown in FIG. 2. Hepa129 (FIGS. 2A, 2B, and 2C) and CT26 (FIGS. 3A, 3B, and 3C) cells were plated onto 96-well plates at a density of 5×10³ cells/well and cultured with Dulbecco's Modifed Eagle Medium (DMEM), 0.1% DMSO, or tricin 7-O-β-D-glucopyranoside analogues at different doses (0.1, 1.0, 10 μg/ml). Twenty four hours (FIGS. 2A and 3A), 48 hr (FIGS. 2B and 3B) and 72 hrs (FIGS. 2C and 3C) after treatment, viability was determined by the MTT assay (Promega Corp., Madison, WI, U.S.). The plates were incubated for 4 h and the absorbance of each well was read at 490 nm. All assays were performed in quadruplicate, and each assay was repeated at least twice.

### Example 9: Acetylated tricin 7-O-β-D-glucopyranoside Exerts a Potent Antitumoral Effect in Mice with CRC that is Superior to Native tricin 7-O-β-D-glucopyranoside.

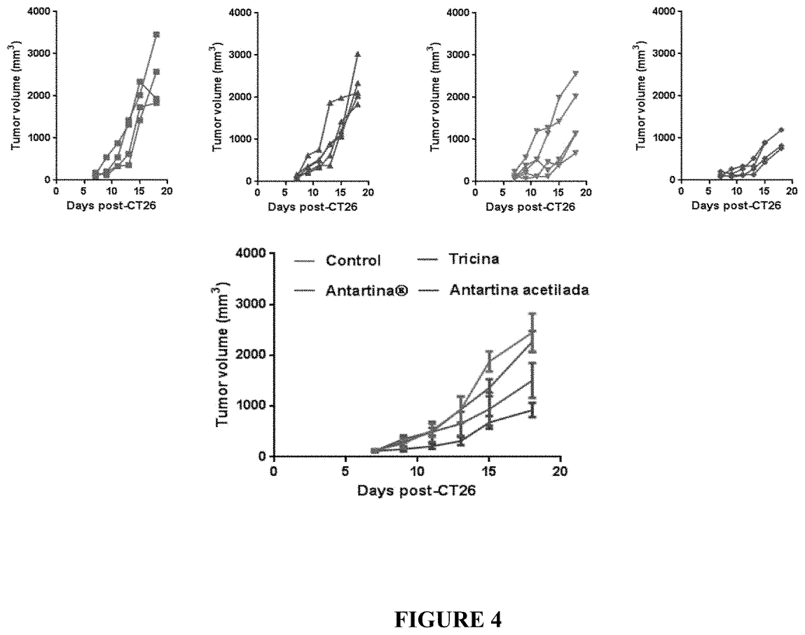

Subcutaneous CRC model: Six-to-eight-week-old male BALB/c mice were used. CT26 cells were injected at a dose of 5×10⁵ cells subcutaneously (s.c.) into the right flank of BALB/c mice (day 0). Tumors were allowed to reach ^{˜}90 mm3 in size before treatment. Animals were distributed in different groups and then treated with 0.1% DMSO in sterile water i.p. (control group, n=5), tricin 7-O-β-D-glucopyranoside (50 mg/kg i.p., day 8, n=5), tricin (50 mg/kg n=8), or acetylated tricin 7-O-β-D-glucopyranoside 50 mg/kg i.p., day 8, n=5), 3 times per week over the course of 3 weeks. (See, FIG. 3, top graphs, left to right: control, tricin 7-O-β-D-glucopyranoside, tricin, and acetylated tricin 7-O-β-D-glucopyranoside. Tumor growth was assessed by caliper measurement. Tumor volume was calculated using the following formula: tumor volume (mm³)=length×(width)²/2. FIG. 4 shows that acetylated tricin 7-O-β-D-glucopyranoside clearly and markedly reduced tumor size at a higher rate than observed for any of the other tested agents.

### Example 10: Antitumoral Effects of Acetylated tricin 7-O-β-D-glucopyranoside in an Orthotopic Hepatocellular Carcinoma Model in Mice

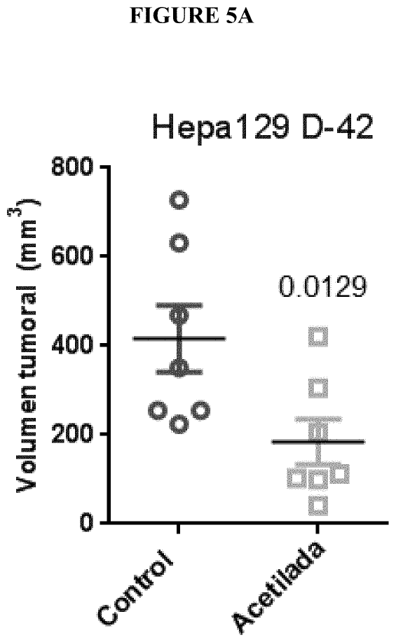

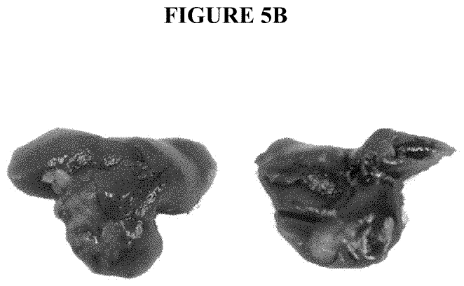

Experimental model of HCC associated with fibrosis: C3H/He mice were treated intraperitoneally (i.p) with 200 mg/kg of thioacetamide (TAA) (Sigma-Aldrich, St. Louis, USA) 3 times a week for 4 weeks. On day 28, mice were anesthetized and orthotopic tumors were established by sub-capsular inoculation of 1.25×10⁵ Hepa129 cells into the left liver lobe by laparotomy (day 0). Five days after tumor implantation, mice were distributed in groups (n=6-8/group) and received: i) saline (control); ii) acetylated tricin 7-O-β-D-glucopyranoside (50 mg/kg, 3 times per week). (See, FIG. 5A). Fifteen days after tumor implantation, mice were sacrificed and tumor volume was quantified. (See, FIG. 5B). Acetylated tricin 7-O-β-D-glucopyranoside markedly reduced the tumor volume.

### Example 11: Tricin 7-O-β-D-glucopyranoside in Combination with Immuno-Gene Therapy (Adenovirus-IL12) shows a Synergistic Antitumoral Effect

In preclinical models of cancer, gene therapy with interleukin 12 (IL-12) has reached unprecedented levels of success when combined with immunotherapy approaches such as gene transfer of other cytokines and/or chemokines, costimulatory molecules, or adoptive cell therapy. (Melero et al., Trends Immunol., 22(3):113-115, 2001).

This is a murine model of CRC (CT26 cells) established in BALB/c mice. Mouse CT26 cells (5×10⁵) were subcutaneously inoculated on day 0. On day 7, when tumors reached a diameter of 5-7 mm, animals were divided into 4 groups: -saline, -tricin 7-O-β-D-glucopyranoside (Antartina) 50 mg/kg, 3 doses per week (intraperitoneally), AdIL12 (adenovirus encoding murine IL-12, dose: 10⁹ TCID50, intratumorally, one dose at day 15 after tumor inoculation), and the combination of tricin 7-O-βD-glucopyranoside ((Antartina) 50 mg/kg, 3 doses per week (intraperitoneally)+AdIL-12 (dose: 10⁹ TCID50, intratumorally, one dose at day 15 after tumor inoculation).

Tumor volume was measured with a caliper 3 times per week.

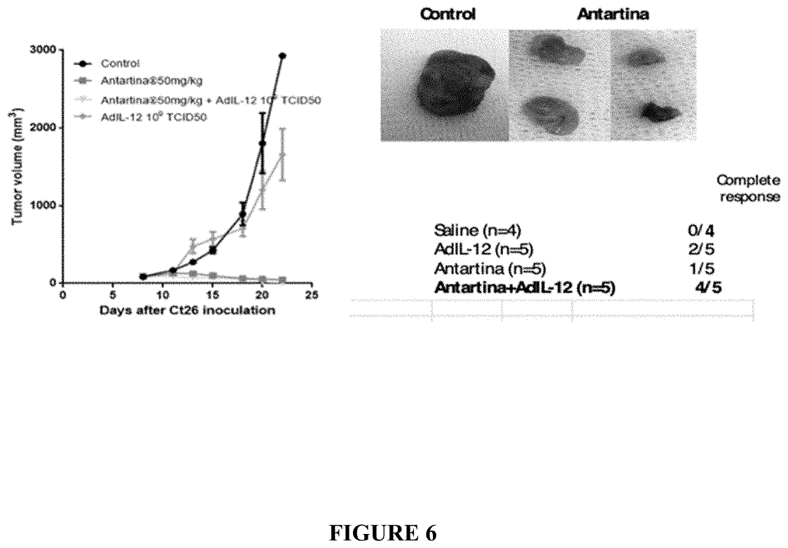

The graph in FIG. 6 shows the growth rate of the tumors for 20 days after the inoculation. Clearly both the 7-O-β-D-glucopyranoside (Antartina)-treatment as well as the treatment with 7-O-β-D-glucopyranoside (Antartina) in combination with AdIL-12 prevented the tumor growth after the inoculation, while the growth continued in control mice (saline) and in mice injected with AdIL12). However, a surprising synergistic antitumor effect was observed when a complete response to the treatments was evaluated: the combination of AdIL-12 and tricin 7-O-β-D-glucopyranoside had eradicated the tumor completely in 4 out of 5 mice. In mice treated with tricin 7-Oβ-D-glucopyranoside alone, 1 out of 5 mice did have no tumor left at the evaluation date. AdIL-12 treatment eradicated tumors in 2 out of 5 mice.

FIG. 6 shows that a synergistic antitumor effect was observed for AdIL-12+tricin 7-O-β-D-glucopyranoside. Complete tumor regressions: saline 0/4, AdIL-12 2/5, Antartina 1/5, and combined therapy: 4/5. (FIG. 6).

It is suggested that the combinatory treatment with tricin 7-O-β-D-glucopyranoside and AdIL-12 is a powerful anticancer treatment that can eradicate colon carcinoma tumors from mammals.

The breadth and the scope of the present disclosure should not be limited by any of the above-described exemplary embodiments, but should be defined only in accordance with the following claims and their equivalents.

1. A method to treat colorectal cancer in a mammal, the method comprising a step of administering AdIL-12 and tricin 7-O-β-D-glucopyranoside to a subject having at least one colorectal tumor.

2. The method of claim 1, wherein AdIL-12 is administered intratumorally and tricin 7-O-β-D-glucopyranoside peritoneally.

3. A method to eradicate a colorectal tumor in a mammal, the method comprising combinatory treatment with AdIL-12 and tricin 7-O-β-D-glucopyranoside.

4. An anticancer treatment for treating colorectal tumors in mammals, the treatment comprising AdIL-12 and tricin 7-O-β-D-glucopyranoside.

5. The treatment of claim 4, wherein tricin 7-O-β-D-glucopyranoside 50 mg/kg is administered intraperitoneally for at least 3 doses per week and one dose of 10⁹ TCID50 of AdIL-12 is administered intratumorally.

6. The treatment of claim 4, effective in preventing or inhibiting growth of a colorectal tumor and in eradication of colorectal tumors.

7. The treatment of claim 5, resulting in at least 80% of eradication of the tumors.
